(19) United States
(12) Patent Application Publication
(10) Pub. No.: US 2026/0315182 A1
(43) Pub. Date: Oct. 15, 2026

(54) IMPLANT WITH FIDUCIAL MARKERS

(71) Applicant: Cornell University, Ithaca, NY (US)

(72) Inventor: Rache Simmons, New York, NY (US)

(21) Appl. No.: 19/467,689

(22) Filed: Feb. 2, 2026

Related U.S. Application Data
(60) Provisional application No. 63/753,091, filed on Feb. 3, 2025.

(51) Int. Cl. A61B 90/00; A61B 6/00; A61B 6/50

(52) U.S. Cl. CPC A61B 90/39; A61B 6/481; A61B 6/502; A61B 2090/3908; A61B 2090/3966

(57) ABSTRACT

An implant includes a strand, a plurality of bodies, and at least one radiopaque marker. Each of the plurality of bodies is supported by the strand, and each of the plurality of bodies is substantially radiolucent. The at least one radiopaque marker is configured to mark the implant in a radiographic image. The at least one radiopaque marker is supported by the strand between and spaced apart from two neighboring bodies of the plurality of bodies.

### CROSS-REFERENCE TO RELATED APPLICATION

This patent application claims the benefit of and priority to U.S. Patent Application No. 63/753,091, filed Feb. 3, 2025, which is hereby incorporated by reference herein in its entirety.

### BACKGROUND

Patients with breast cancer may elect to undergo treatment to treat a lump or tumor. Treatment options include a mastectomy, a lumpectomy, radiation, and/or chemotherapy depending on several factors, such as size and location of the tumor. In some cases, patients may undergo multiple treatments to improve likelihood of survival. For example, a patient may undergo a lumpectomy procedure, followed by focused radiation therapy. It may also be desirable to monitor tissue of the breast at or near the surgical site, such as at a cavity or void created by removal of a tumor.

### BRIEF DESCRIPTION OF THE DRAWINGS

The detailed description particularly refers to the accompanying figures in which:

FIG. 1 illustrates a front view of a lumpectomy procedure that creates a cavity in breast tissue of the patient due to removal of tissue;

FIG. 2 illustrates a perspective view of an implant configured to be implanted into the cavity of FIG. 1, the implant including a strand, a plurality of bodies coupled to the strand, and at least one marker coupled to the strand and configured to mark the cavity in radiographic imaging;

FIG. 3 illustrates a front view of the cavity of FIG. 1 filled with the implant of FIG. 2;

FIG. 4 illustrates a perspective view of one of the plurality of bodies of FIG. 2, the body including a bead that defines a channel for the strand to extend through;

FIG. 5 illustrates a perspective view of the marker of FIG. 2, the marker defining at least one bore for the strand to extend through;

FIG. 6 illustrates a cross-sectional view of another embodiment of a body for use with the implant of FIG. 2, the body including a bead that defines a channel for the strand to extend through and at least one chamber to decrease a weight of the bead;

FIG. 7 illustrates a perspective view of another embodiment of an implant configured to be implanted into the cavity of FIG. 1, the implant including a strand, a plurality of non-spherical bodies coupled to the strand, and at least one marker coupled to the strand and configured to mark the cavity in radiographic imaging;

FIG. 8 illustrates an enlarged perspective view of the implant of FIG. 7 showing the at least one marker coupled to the strand;

FIG. 9 illustrates a perspective view of another marker for use with the implant of FIG. 2 or FIG. 7; and

FIG. 10 illustrates a schematic view of a method.

### DETAILED DESCRIPTION

In a lumpectomy operation, a tumor or a cancerous mass is removed from a patient's breast through resection from healthy breast tissue. The lumpectomy procedure may result in a cavity within the patient's breast that becomes a target for subsequent radiation therapy to treat any remaining cancer cells post-resection. In some instances, the cavity may form a post operative deformity that conventional breast implants are not designed to fill.

Implants may be used to fill the cavity, serving dual purposes as both radiation therapy markers (i.e., fiducials) and breast reconstruction aids. Conventional implants can cause patient discomfort, particularly due to the selection of hard or sharp materials included in conventional implants. Conventional implants can also break down in a patient's body, forming hard or sharp fragments that may cause additional discomfort to the patient. Radiopaque markers used as fiducials can also be constructed of hard materials, creating further discomfort when used in large quantities due to their sharpness and rigidity. Monitoring of the breast at or near the surgical site may occur over the lifetime of the patient, making proper marking of the area of interest and comfort of the implant more important.

The present disclosure relates to an implant designed to fill a cavity formed due to a resection procedure that is both flexible and comfortable to the patient. The implant is formed as a strand, which may also be referred to as a strand of pearls, incorporating bodies and fiducial markers distributed along the length of the strand. The strand of pearls design helps to enable efficient packing into the post-lumpectomy cavity and can be adjusted to accommodate varying cavity volumes. The flexible nature of the implant allows it to conform to diverse cavity shapes, adapting to each individual patient's anatomy.

In some embodiments, the bodies of the implant are constructed from non-bioresorbable material with a low durometer, chosen to help mimic the softness of natural breast tissue and increase patient comfort. Fiducial markers are positioned at intervals along the strand, providing reference points for radiation therapy with their quantity limited to prevent patient discomfort. The implant offers a flexible and customizable solution to the surgeon, configurable to various lengths to accommodate individual patient circumstances.

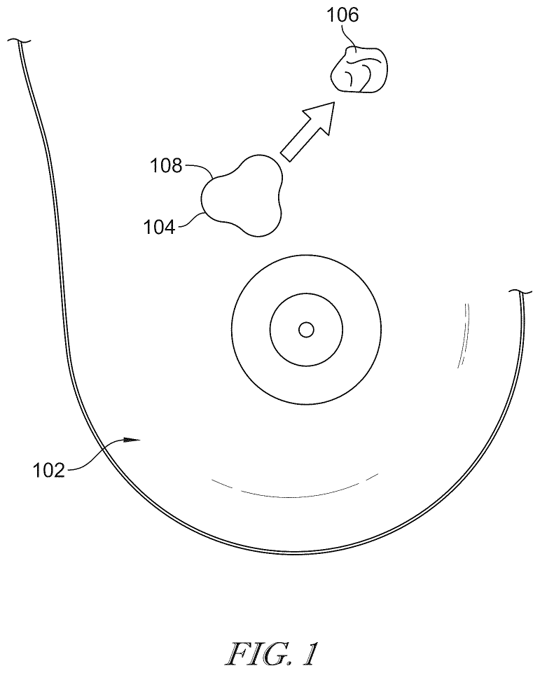

FIG. 1 illustrates a front view of a human breast 102 in a lumpectomy procedure. As shown in FIG. 1, an incision 104 is created in the human breast 102. The tumor 106 is removed from the human breast 102 via the incision 104. Due to the removal of the tumor 106, a cavity 108 is formed in the breast 102.

In a lumpectomy operation to remove the tumor 106, the incision 104 is created on the breast 102, such as by using a scalpel. Using the incision 104 to access the tumor 106, the tumor 106 can be resected and removed from the breast 102, as shown in FIG. 1. During removal of the tumor 106 from the breast 102, some surrounding breast tissue can also be resected to create a margin around the tumor 106. Removal of the tumor 106 from the breast 102 (and margin creation) creates the cavity 108 within the breast 102. The cavity 108 may be a target for future radiation therapy where radiation can be directed at the cavity 108 to treat any cancerous (or pre-cancerous) cells that may remain following resection and removal of the tumor 106. It can be helpful to fill the cavity 108 with one or more fiducial markers to act as a radiopaque target for future radiation therapy of the cavity 108 and the surrounding area. The one or more fiducial markers for radiotherapy or radiographic imaging are implanted or placed within or on the body to serve as a reference point during imaging or treatment procedures. The one or more fiducial markers are used to improve the accuracy of targeting specific areas, such as tumors, in radiotherapy or diagnostic imaging (e.g., X-ray, computed tomography (CT), magnetic resonance imaging (MRI), or ultrasound).

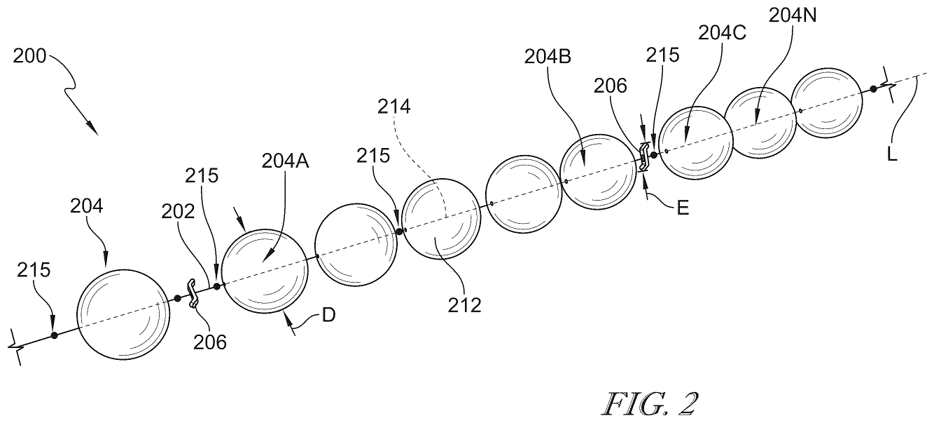

FIG. 2 illustrates a perspective view of an implant 200 configured to be implanted into the cavity 108. The implant 200 includes a strand 202, a plurality of bodies 204, and at least one marker 206. The strand 202 extends along a longitudinal axis L. The plurality of bodies 204 and the at least one marker 206 are coupled to the strand 202. Each of the plurality of bodies 204 is coupled to the strand 202 in spaced apart relation to one another along the longitudinal axis L. The at least one marker 206 is located between two neighboring bodies 204 and spaced apart from the neighboring bodies 204.

In illustrative embodiments, the strand 202 is elongate and flexible. In some embodiments, the strand 202 includes various physical configurations, such as, a braided construction, a twisted configuration, a woven configuration, or the like. In some embodiments, the strand 202 includes surface coatings, such as, but not limited to, a lubricious coating, an antimicrobial coating, a surface texture, a bioactive component, or the like.

The strand 202 is illustratively made of a biocompatible material. In some embodiments, the strand 202 is made of a bioresorbable material such that the strand 202 is configured to be broken down and absorbed by the human body. In some embodiments, the strand 202 is made of a non-bioresorbable material such that the strand 202 permanently remains in the body (unless removed by a healthcare provider). In some embodiments, the strand 202 is made of a combination of a bioresorbable material and a non-bioresorbable material.

In some examples, the strand 202 may be comprised of one or more of a collagen, a glycosaminglycan, a starch, a silk, an alginate, a chitin, a chitosan, a polylactic acid, a polyglycolic acid, a polyanhydride, a polycaprolactone, a poly(hydroxybutyrate), a poly(hydroxuvalerate), a polylactic acid, a polyglactin, a polydioxanone, catgut, or the like. In an example, the strand 202 may be a suture. In some examples, the suture can be made of one or more of polypropylene, cotton, silk, nylon, Vicyrl (copolymer of glycolide and lactide), gold, silver, platinum, cobalt, chromium, or the like.

The plurality of bodies 204 may include any number of bodies 204A, 204B, 204C, 204N. In some embodiments, the plurality of bodies 204 is omitted and the implant 200 alternatively includes a singular body 204. The plurality of bodies 204 is configured to mimic the characteristics of natural breast tissue, providing tactile feedback and natural movement. The biomimetic property of the plurality of bodies 204 helps to increase patient comfort and aesthetic outcome following surgical procedures such as a breast surgery or lumpectomy.

Each of the plurality of bodies 204 is supported by the strand 202, as shown in FIG. 2. The flexibility of the strand 202 enables the longitudinal axis L to adapt and conform to various orientations and positions within the cavity 108. The flexibility of the strand 202 allows for multiple spatial configurations and arrangements of the plurality of bodies 204 thereby allowing for adaptable positioning and orientation of the implant 200.

The at least one marker 206 is configured to mark the cavity 108 in a radiographic image following implantation of the implant 200 within the cavity 108. In this way, the marker 206 can be configured to act as fiducial marker for radiation therapy. The at least one marker 206 is illustratively radiopaque, wherein radiopaque refers to a property of a material or object that makes the material visible in imaging modalities like X-rays, CT scans, or MRI images by blocking or attenuating X-ray radiation. The at least one marker 206 serves as a reference point during radiation therapy by visibly appearing in medical imaging (e.g., X-rays, CT scans, or MRI images), allowing a healthcare provider to target radiation treatment to the cavity 108 where cancer cells may remain. The at least one marker 206 enables accurate alignment and position of a radiation beam during a therapy session, ensuring that radiation is delivered to the intended treatment area while minimizing exposure to surrounding healthy tissue.

As shown in FIG. 2, the at least one marker 206 is located between two of the plurality of bodies 204. In some embodiments, the at least one marker 206 includes two or more markers 206 distributed along the longitudinal length L of the strand 202. For example, two markers 206 can be connected to the strand 202, separated by one, two, three, four, five, or more bodies 204. The spacing of the markers 206 between the bodies 204 can repeat along the longitudinal length L of the strand 202. The distribution pattern of the one or more markers 206 relative to the bodies 204 can follow a uniform spacing arrangement (e.g., after every one, two, three, four, or five bodies 204), or a variable spacing pattern in which the number of intervening bodies 204 between markers 206 varies randomly or in a predetermined sequence.

In some embodiments, the bodies 204 and/or the one or more markers 206 are fixed to the strand 202 such that the bodies 204 and/or the one or more markers 206 do not move along the longitudinal length L of the strand 202. In some embodiments, the bodies 204 and/or the one or more markers 206 are connected to the strand 202 by methods such as bonding, adhering, fastening, or the like.

In some embodiments, the bodies 204 and/or the one or more markers 206 are coupled to the strand 202 such that the bodies 204 and/or the one or more markers 206 are free to slide or translate along the longitudinal length L of the strand 202. Such movement of the bodies 204 and/or the one or more markers 206 enable the implant 200 to be shaped and arranged to a shape or conformation that matches the cavity 108. In some embodiments, the strand 202 includes one or more knots 215 engageable with the bodies 204 and/or the one or more markers 206 to limit translation of the bodies 204 and/or the one or more markers 206 along the strand 202. Any of the knots can be any mechanism configured to secure the bodies 204 and the markers 206 such as one or more knots, fasteners, adhesives, or the like. In some embodiments, some of the bodies 204 and/or the markers 206 are fixed to the strand 202 and others of the bodies 204 and/or the markers 206 are free to slide or translate along the strand 202. In some embodiments, a knot 215 is located between each of the bodies 204 and the markers 206.

Each of the bodies 204 define a diameter D, as shown in FIG. 2. The marker 206 defines a length E. In illustrative embodiments, the length E of the marker 206 is less than the diameter D of the bodies 204. For example, the length E is less than 50% of the diameter D, less than 45% of the diameter D, less than 40% of the diameter D, less than 35% of the diameter D, less than 30% of the diameter D, less than 25% of the diameter D, less than 20% of the diameter D, or less than 15% of the diameter D. In some embodiments, the diameter D is about 5 millimeters to 15 millimeters. In some embodiments, the diameter D is 10 millimeters. In some embodiments, the length E is about 0.5 millimeters to 1.5 millimeters. The size of the marker 206 is smaller than the bodies 204 because the materials of the markers 206 are of greater structural rigidity compared to the material of the bodies 204. The size of the marker 206 is designed to be minimally invasive, to reduce patient discomfort, and to reduce tissue disruption.

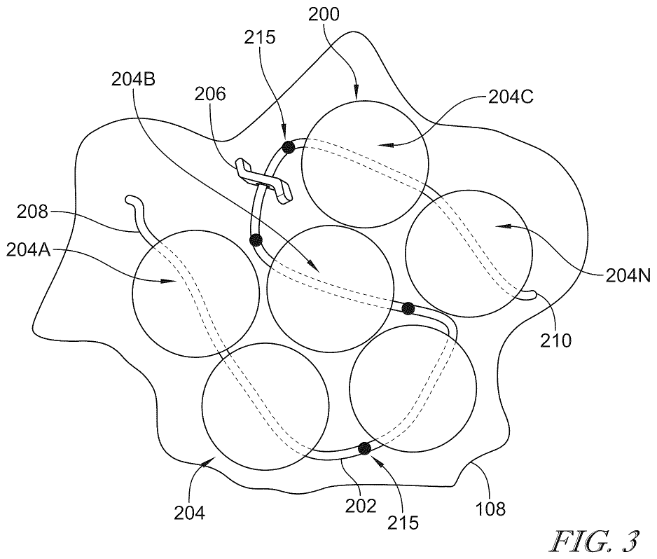

FIG. 3 illustrates a front view of the cavity 108 filled with the implant 200. Due to the flexibility of the strand 202, the implant 200 can match a shape and a volume of the cavity 108 to be filled.

In some examples, the implant 200 can be customized to fit the size of the cavity 108 by cutting or breaking along the strand 202. For example, a healthcare provider can determine an appropriate number of bodies 204 to achieve cavity filling, and the healthcare provider can trim or break the strand 202 accordingly. The implant 200 can be adjusted (e.g., via trimming or breaking) to a final length ranging from about 10 centimeters to 50 centimeters, for example, about 10 centimeters, about 20 centimeters, about 30 centimeters, about 40 centimeters, or about 50 centimeters. In some embodiments, the implant 200 can be more than 50 centimeters long for insertion into the cavity 108. The implant 200, when cut or broken to an appropriate length for insertion into the cavity 108, includes at least one marker 206, which serves as a fiducial maker for a subsequent radiation therapy procedure

When the length of the implant 200 has been determined, a first free end 208 of the implant 200 can be secured to tissue of the cavity 108. The implant 200 can be arranged and packed to assume a variety of configurations to at least partially fill the cavity 108. The flexibility of the strand 202 allows the implant 200 to be manipulated or positioned into various configurations, including folded, coiled, or layered arrangements within the cavity 108, thereby facilitating at least partial filing of the cavity 108. The adaptable packing capability of the implant 200 helps a healthcare provider to customize the implant 200 to a patient's unique anatomy. The ability of the implant 200 to be arranged to conform to various shapes helps keep the cavity 108 open post-operatively thereby reducing breast indentations, which can improve aesthetics of the breast following a lumpectomy procedure. Keeping the cavity 108 open post-operatively can also help to promote fluid intake into the cavity 108 thereby promoting healing and tissue ingrowth to help permanently fill the cavity 108. The shaping, arranging, packing, and insertion of the implant 200 may be done by hand and/or using forceps by a healthcare provider.

After packing the implant 200 into the cavity 108, a second free end 210 of the implant 200 can be anchored to the tissue of the cavity 108 to secure the implant 200 in position. The anchoring of the first free end 208 and the second free end 210 helps to maintain proper placement of the implant 200 within the cavity 108 throughout the treatment period.

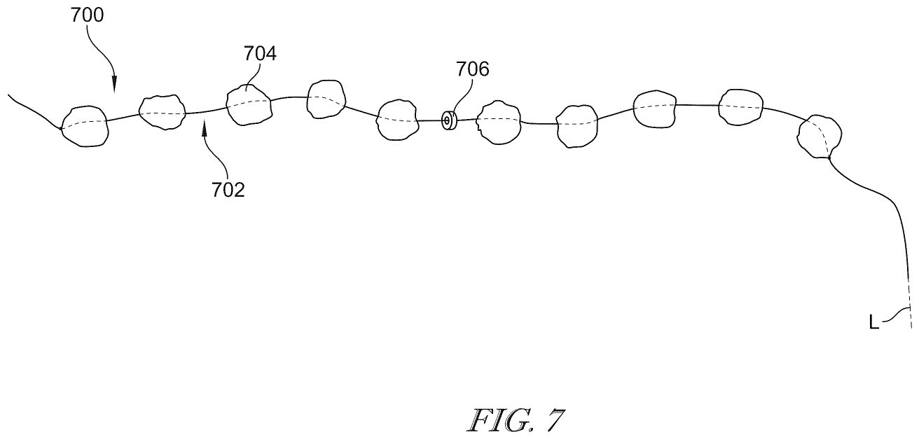

FIG. 4 illustrates a perspective view of one of the bodies 204 included in the implant 200. Each of the bodies 204 includes a bead 212 and a channel 214. The channel 214 extends through the bead 212. In illustrative embodiments, the bead 212 is a sphere. In some embodiments, the bead 212 has a non-spherical shape (see FIG. 7), such as, an ovoid, an oblate spheroid, a prolate spheroid, a cube, a dodecahedron, an icosahedron, or the like. Use of a rounded shape without a sharp edge can help to reduce tactile detection of the implant 200 following implantation in the cavity 108. In some embodiments, the body 204 is solid (except for the channel 214).

In some embodiments, the channel 214 extends through a center O of the bead 212. In some embodiments, the channel 214 is offset from the center O. The strand 202 extends through the channel 214 to connect the bead 212 to the strand 202. The channel 214 can be formed by a variety of manufacturing process, such as, but not limited to, extrusion, insert molding, two-shot molding, injection molding, machining, additive manufacturing, or the like.

The bead 212 is formed of biocompatible materials. In some embodiments, the bead 212 is formed of non-bioresorbable materials. In some embodiments, the bead 212 is formed of bioresorbable materials. In some embodiments, the bead 212 is formed of polyethylene, polyurethane, silicone, hydrogels, thermoplastic urethane, thermoplastic elastomer, any suitable combination of the same, or the like. The material of the bead 212 has a Shore 00 durometer of 20 or less to provide a soft tissue-like feel that can aid in patient comfort while maintaining structural integrity for cavity filling and marker positioning.

In illustrative embodiments, the material of the bead 212 is radiolucent, allowing radiation to pass through with minimal interference or attenuation during medical imaging and treatment procedures. The radiolucency of the bead 212 enables a healthcare provider to observe tissue in the cavity 108 using radiographic imaging techniques without obstruction by the bead 212.

In some embodiments, the material of the bead 212 is radiation resistant such that its structural and mechanical properties, when exposed to therapeutic radiation doses, remain substantially the same without significant degradation or alteration of its physical characteristics due to the therapeutic radiation. In an example, the bead 212 is manufactured from a thermoplastic material, for example, Medalist MD-16120G NAT from Teknor Apex (Pawtucket, RI).

In some embodiments, the bead 212 is coated with an antineoplastic drug coating such that, following implantation, the antineoplastic drug coating can interact with cancerous cells that may persist following a lumpectomy or other procedure that may form the cavity 108. Such an interaction can provide or create a local tissue infusion within the margins of the tissue surrounding the cavity 108 to amplify destruction of any residual tumor cells in the surrounding tissue. The antineoplastic drug coating may include one or more of Tamoxifen, an Aromatase inhibitor, Hecrceptin, or the like.

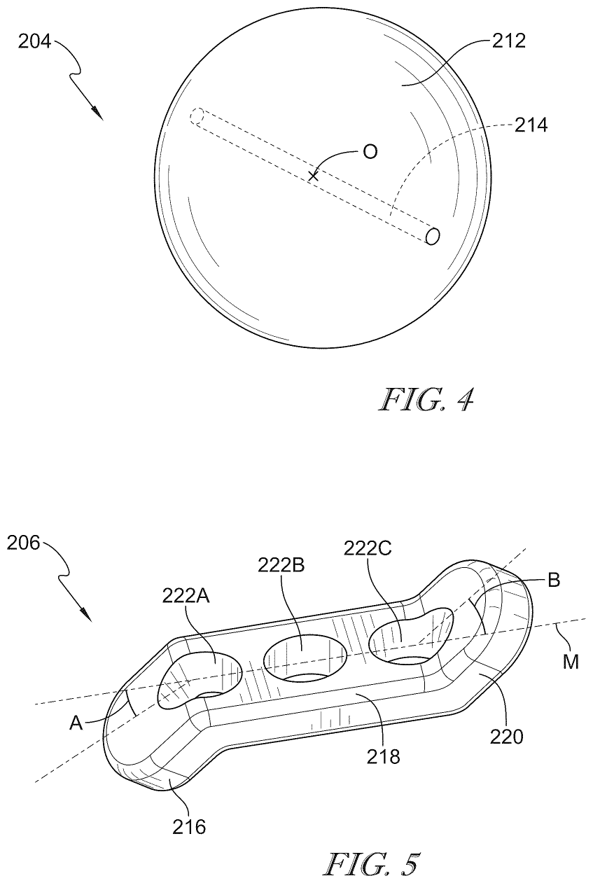

FIG. 5 illustrates a perspective view of one of the markers 206. The marker 206 extends along a longitudinal axis M. The marker 206 includes a first portion 216, a second portion 218, and a third portion 220. The second portion 218 extends along the longitudinal axis M, and the first and third portions 216, 220 are angled relative to the longitudinal axis M. The first and third portions 216, 220 are angled in opposite directions, as shown in FIG. 5. For example, the first portion 216 is coupled to an end of the second portion 218 to extend away therefrom in a first direction, and the third portion 220 is coupled to an opposite end of the second portion 218 to extend away therefrom in a second direction opposite the first direction.

The edges of the marker 206 are curved and smooth and free from angular, sharp, or abrupt transitions to prevent poking or stabbing the patient within the cavity 108. The marker 206 includes one or more bores 222A, 222B, 222C in the first portion 216, in the second portion 218, or in the third portion 220. For example, the second bore 222B extends through the second portion 218 perpendicular to the longitudinal axis M. The first bore 222A extends through the first portion 216 and the second portion 218 such that the first bore 222A follows the angle of the marker 206. The third bore 222C extends through the second portion 218 and the third portion 220 such that the third bore 222C follows the angle of the marker 206. In other words, the bores 222A, 222C straddle two of the first portion 216, the second portion 218, and the third portion 220.

The strand 202 extends through at least one of the bores 222A, 222B, 222C, as shown in FIG. 2. The bores 222A, 222B, 222C also provide weight reduction of the marker 206. The shape of the marker 206 provides a distinctive shape for radiographic imaging or the like.

As shown in FIG. 5, the first portion 216 is connected to the second portion 218 at an angle A, and the third portion 220 is connected to the second portion 218 at an angle B (both angles with reference to the longitudinal axis M of the second portion 218). The angles A, B may be acute angles, obtuse angles, or right angles. For example, the angle A and the angle B can be acute angles; the angle A can be an obtuse angle, the angle B can be an acute angle; the angle A can be an acute angle, the angle B can be an obtuse angle; or the angle A and the angle B can be obtuse angles.

The marker 206 is supported by the strand 202, and the strand 202 extends through one or more of the bores 222A, 222B, or 222C of the marker 206. For example, the strand 202 may extend through the bore 222B, as shown in FIGS. 2 and 3. The marker 206 may be engaged by one or more knots on the strand 202 to limit translation of the marker 206 along the strand 202. In some embodiments, the marker 206 is supported by the strand 202 by threading and fastening the strand 202 through one or more of the bores 222A, 222B, 222C and around the marker 206 to limit translation of the marker 206 along the strand 202.

In some embodiments, the marker 206 can take on a variety of shapes to generate distinct radiographic signatures that enhance identification of the marker 206 during an imaging procedure. The marker 206 can take on shapes, such as, a dumbbell shape, a coil shape, a helix shape, an annular shape, a spring shape, a twisted shape, a stent shape, a weave shape, a cylinder shape, a T-shape, a letter shape, a number shape, or the like. In some embodiments, the implant 200 includes one or more markers 206 of one or more different shapes to distinguish different types of implants following implantation.

In some embodiments, the marker 206 is made of a biocompatible and radiopaque material such as, but not limited to, titanium, a titanium alloy, polyether ether ketone, a stainless steel alloy, a cobalt-chromium alloy, a ceramic, a polymer, or the like.

In some embodiments, the implant 200 includes forty bodies 204 and seven markers 206. In some embodiments, the markers 206 are spaced apart with five bodies 204 between each of the markers 206. The strand 202 may be cut, trimmed, or broken by the healthcare provider to the desired length and number of bodies 204.

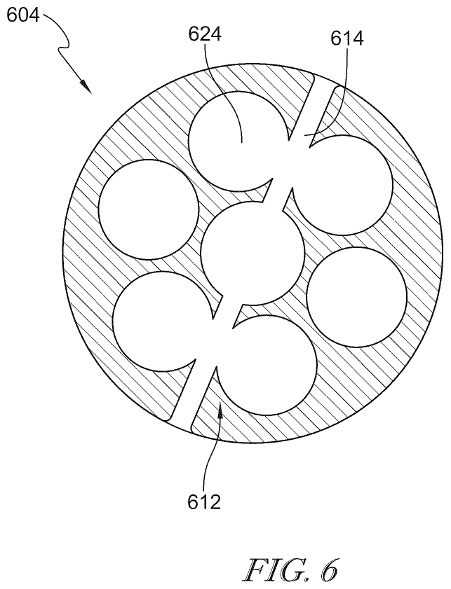

FIG. 6 illustrates a cross-sectional view of another embodiment of a body 604 for use with the implant 200. The body 604 is substantially similar to the body 204 shown in FIGS. 2-4 and described herein. Accordingly, similar reference numbers in the 600 series indicate features that are common between the body 204 and the body 604. The description of the body 204 is incorporated by reference to apply to the body 604, except in instances when it conflicts with the specific description and drawings of the body 604.

In some embodiments, the implant 200 includes one or more bodies 604 and one or more bodies 204 (i.e., the implant 200 includes both bodies 204, 604). In some embodiments, the implant 200 includes only the bodies 604. In some embodiments, the implant 200 includes only the bodies 204.

The body 604 includes a bead 612, as shown in FIG. 6. The bead 612 is formed to define a channel 614 and at least one chamber 624. Similar to the bead 212, the bead 612 can take a variety of shapes and materials discussed above. The channel 614 extends through the bead 612 to receive the strand 202 therein.

The at least one chamber 624 is a void formed in the bead 612 that reduces the density and amount of material in the bead 612, thereby decreasing the overall weight of the implant 200. The reduction of weight can reduce the physical burden on the patient's anatomy thereby reducing stress on surrounding tissues and improving patient comfort. In some embodiments, the at least one chamber 624 is positioned at a center of the bead 612. In some embodiments, the at least one chamber 624 is positioned at a perimeter of the bead 612. In some embodiments, the at least one chamber 624 includes multiple chambers 624 that are spaced apart from one another. In some embodiments, the channel 614 extends through one or more of the chambers 624.

FIG. 7 illustrates another implant 700. The implant 700 is substantially similar to the implant 200 shown in FIGS. 2 and 3 and described herein. Accordingly, similar reference numbers in the 700 series indicate features that are common between the implant 200 and the implant 700. The description of the implant 200 is incorporated by reference to apply to the implant 700, except in instances when it conflicts with the specific description and drawings of the implant 700.

The implant 700 includes a strand 702, a plurality of bodies 704, and at least one marker 706, as shown in FIG. 7. The strand 702 extends along a longitudinal axis L. The plurality of bodies 704 and the at least one marker 706 are coupled to the strand 702. Each of the plurality of bodies 704 is coupled to the strand 702 in spaced apart relation to one another along the longitudinal axis L. The at least one marker 706 is located between two neighboring bodies 704 and spaced apart from the neighboring bodies 704.

The plurality of bodies 704 may include any number of bodies 704. Each of the plurality of bodies 704 is supported by the strand 702, as shown in FIG. 7. The flexibility of the strand 702, as shown in FIG. 7, enables the longitudinal axis L to adapt and conform to various orientations and positions within the cavity 108.

Each of the plurality of bodies 704 is illustratively a non-spherical shape. In some embodiments, the implant 700 includes one or more bodies 704 that vary in size and/or shape.

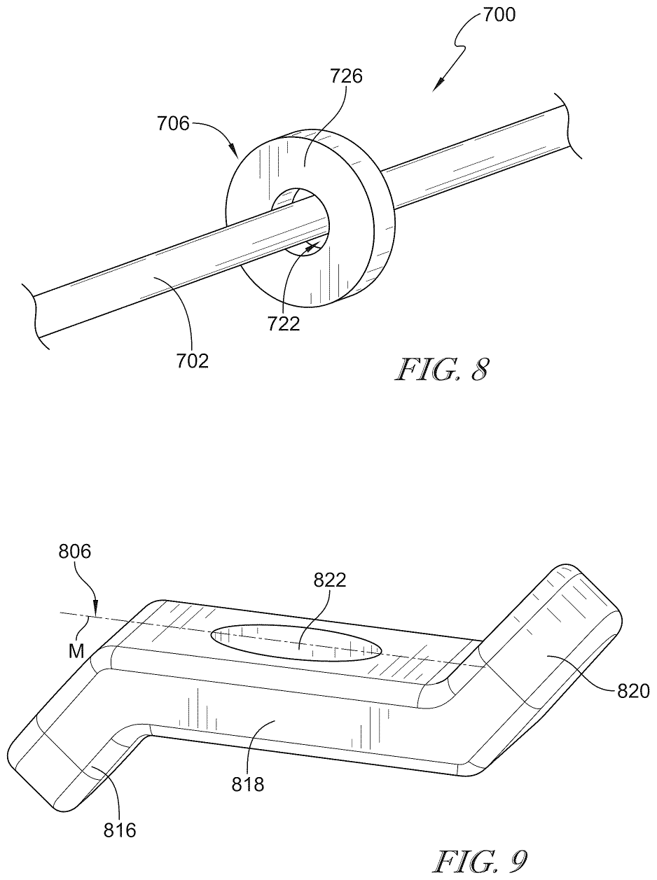

In some embodiments, the marker 706 is a washer, as shown in FIG. 8. In some embodiments, the implant 200 includes the marker 706.

The marker 706 includes an annular body 726 that defines a bore 722, as shown in FIG. 8. The strand 702 is threaded through the bore 722. In some embodiments, the marker 706 is supported by the strand 702 by threading and fastening the strand 702 through the bore 722 and around the annular body 726 to limit translation of the marker 706 along the strand 702 (i.e., tying or knotting the strand 702 around the annular body 726).

FIG. 9 illustrates another embodiment of a marker 806 for use with the implant 200, 700. The marker 806 is substantially similar to the marker 206 shown in FIGS. 2, 3, and 5 and described herein. Accordingly, similar reference numbers in the 800 series indicate features that are common between the marker 206 and the marker 806. The description of the marker 206 is incorporated by reference to apply to the marker 806, except in instances when it conflicts with the specific description and drawings of the marker 806.

In some embodiments, the implant 200, 700 may include a combination of the markers 206, 706, 806. The marker 806 extends along a longitudinal axis M. The marker 806 includes a first portion 816, a second portion 818, and a third portion 820. The second portion 818 extends along the longitudinal axis M, and the first and third portions 816, 820 are angled relative to the longitudinal axis M. The first and third portions 816, 820 are angled in opposite directions, as shown in FIG. 9. For example, the first portion 816 is coupled to an end of the second portion 818 to extend away therefrom in a first direction, and the third portion 820 is coupled to an opposite end of the second portion 818 to extend away therefrom in a second direction opposite the first direction.

The edges of the marker 806 are curved and smooth and free from angular, sharp, or abrupt transitions to prevent poking or stabbing the patient within the cavity 108. The marker 806 includes a bore 822 extending through the second portion 818 perpendicular to the longitudinal axis M.

The strand 202, 702 extends through the bore 822. The bore 822 also provides weight reduction of the marker 806. The shape of the marker 806 provides a distinctive shape for radio imaging, or the like.

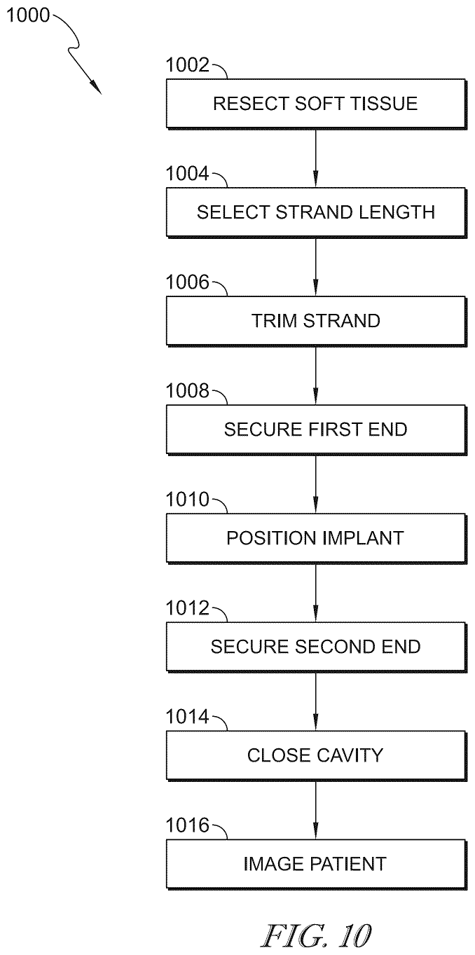

FIG. 10 illustrates a schematic view of a method 1000 in accordance with at least one example of this disclosure. The method 1000 may be a method of implanting an implant 200, 700. The steps or operations of the method 1000 are illustrated in a particular order for convenience and clarity; many of the discussed operations can be performed in a different sequence or in parallel without materially impacting other operations. The method 1000 as discussed includes steps or operations performed by multiple different actors, devices, or systems. It is understood that subsets of the sets or operations discussed in the method 1000 can be performed by one or more actors, devices, or systems, and each step, operation, set of steps, or set of operations may constitute an independent process or method.

The method 1000 begins at step 1002. At step 1002, soft tissue is resected in a lumpectomy procedure to create a volume defect (i.e., the cavity 108).

At step 1004, a length of the strand 202, 702 of the implant 200, 700 is selected based on a size of the cavity 108. The strand 202, 702 is elongate and flexible. For example, the strand 202, 702 of the implant 200, 700 may be provided at a relatively long length, and a length of the strand 202, 702 may be selected. In some embodiments, more than one implant 200, 700 with different strand lengths may be provided, and the implant 200, 700 may be chosen based on the desired length.

At step 1006, the strand 202, 702 is cut, trimmed, or broken, if desirable, to reduce the strand length. The strand 202, 702 includes the marker 206, 706, 806 defining the bore 222A, 222B, 222C, 722, 822 to support the marker 206, 706, 806 on the strand 202, 702. The marker 206, 706, 806 is illustratively located between at least two of the plurality of bodies 204, 604, 704. For example, as shown in FIG. 2, the marker 206 is located between the bodies 204B, 204C.

At step 1008, the first free end 208 of the strand 202, 702 is secured to tissue at a first location that at least partially forms the cavity 108. For example, as shown in FIG. 3, the first free end 208 is secured to tissue of the cavity 108.

At step 1010, the strand 202, 702 and the plurality of bodies 204, 604, 704 are positioned to conform to a shape of the cavity 108. The implant 200, 700 is positioned within the cavity 108 to substantially fill a volume of the cavity 108 and to mark the cavity 108 with the marker 206, 706, 806. For example, as shown in FIG. 3, the implant 200 comprising the strand 202 and the bodies 204 is arranged to conform to the shape of the cavity 108. The strand 202, 702 and the plurality of bodies 204, 604, 704 are configured to flex within the cavity 108 after implantation.

At step 1012, the second free end 210 of the strand 202, 702 is secured to the tissue at a second location that at least partially forms the cavity 108. For example, as shown in FIG. 3, the second free end 210 is secured to tissue of the cavity 108. At step 1014, the cavity 108 is closed. At step 1016, the patient is imaged. In step 1016, electromagnetic waves pass through the plurality of bodies 204, 604, 704 so that tissues of the patient are not substantially obstructed by the plurality of bodies 204, 604, 704 and electromagnetic waves are blocked with the marker 206, 706, 806 to mark the cavity 108 on imaging of the patient. In this way, the healthcare provider can locate the implant 200, 700 and, thus, the cavity 108. The healthcare provider can then administer radiation therapy.

Though the methods and systems herein are discussed above with respect to lumpectomies performed in human breasts, the implants, systems, and methods can be used in any other procedure where a surgical procedure leaves a crater, a dent, a divot, or a recessed cavity in the body, such as, but not limited to, the removal of tumors, cysts, lipomas, or dermoid cysts.

1. An implant configured to be implanted into a patient, the implant comprising:
a strand extending along a longitudinal axis, the strand being elongate and flexible;
a plurality of bodies supported by the strand along the longitudinal axis thereof, each of the plurality of bodies being substantially radiolucent; and
at least one radiopaque marker configured to mark the implant in a radiographic image, the at least one radiopaque marker supported by the strand along the longitudinal axis thereof between and spaced apart from two neighboring bodies of the plurality of bodies, the at least one radiopaque marker defining a bore that extends therethrough to receive the strand therein.

2. The implant of claim 1, wherein each body of the plurality of bodies includes a bead that defines a channel extending therethrough to receive the strand therein to secure the body to the strand.

3. The implant of claim 2, wherein the channel extends through a center of the bead.

4. The implant of claim 2, wherein the bead of each body of the plurality of bodies defines at least one chamber therein to decrease a weight of the bead.

5. The implant of claim 1, wherein the strand includes one or more knots engageable with one or more of the plurality of bodies and the at least one radiopaque marker to limit translation of the one or more of the plurality of bodies and the at least one radiopaque marker along the strand.

6. The implant of claim 1, wherein each of the plurality of bodies is formed of a thermoplastic elastomer.

7. The implant of claim 6, wherein the thermoplastic elastomer has a Shore 00 durometer of 20 or less.

8. The implant of claim 1, wherein each of the plurality of bodies defines a diameter, and wherein the at least one radiopaque marker defines a length that is smaller than the diameter.

9. The implant of claim 8, wherein the length is less than 25 percent of the diameter.

10. The implant of claim 8, wherein the length is less than 50 percent of the diameter.

11. The implant of claim 1, wherein the at least one radiopaque marker includes a first portion, a second portion coupled to the first portion, and a third portion coupled to the second portion opposite the first portion, and wherein the first portion and the third portion are angled relative to the second portion.

12. The implant of claim 11, wherein the bore extends through the second portion.

13. The implant assembly of claim 1, wherein the at least one radiopaque marker includes a first radiopaque marker and a second radiopaque marker spaced apart from the first radiopaque marker, and wherein five bodies of the plurality of bodies are located between the first radiopaque marker and the second radiopaque marker along the longitudinal axis of the strand.

14. The implant assembly of claim 1, wherein the at least one radiopaque marker includes an annular body that defines the bore.

15. A method of marking a body cavity of a patient with an implant, the method comprising:
selecting a strand length of a strand of the implant based on a size of the body cavity;
positioning the implant in the body cavity to substantially fill a volume of the body cavity, the implant including a plurality of bodies supported by the strand and at least one radiopaque marker supported by the strand, the at least one radiopaque marker located between and spaced apart from two neighboring bodies of the plurality of bodies;
closing the cavity;
imaging the patient; and
passing electromagnetic waves through the plurality of bodies so that tissues of the patient are not substantially obstructed by the plurality of bodies and blocking electromagnetic waves with the at least one radiopaque marker to mark the body cavity on imaging of the patient.

16. The method of claim 15, further comprising securing a first free end of the strand to tissue at a first location that at least partially forms the body cavity.

17. The method of claim 16, further comprising, after securing the first free end, arranging the strand and the plurality of bodies in the body cavity to conform to a shape of the body cavity.

18. The method of claim 17, further comprising, after arranging the strand and the plurality of bodies, securing a second free end of the strand to tissue at a second location that at least partially forms the body cavity.

19. The method of claim 15, wherein selecting the strand length includes selecting a number of bodies of the plurality of bodies based on the size of the body cavity.

20. The method of claim 15, further comprising, after selecting the strand length, reducing the strand length.
